(19) United States
(12) Patent Application Publication
(10) Pub. No.: US 2025/0144366 A1
(43) Pub. Date: May 8, 2025

(54) PUSH CONNECT CATHETER HUB AND RELATED METHODS

(71) Applicant: BARD PERIPHERAL VASCULAR, INC., Franklin Lakes, NJ (US)

(72) Inventors: Zachary Willis, Mesa, AZ (US); Nicholas Peterson, Mesa, AZ (US); Kyle Thomas, Phoenix, AZ (US)

(21) Appl. No.: 18/837,870

(22) PCT Filed: Feb. 11, 2022
(86) PCT No.: PCT/US2022/016097

§ 371 Date: Aug. 12, 2024

(51) Int. Cl. A61M 25/00; A61M 39/10

(52) U.S. Cl. CPC A61M 25/0097; A61M 25/0026; A61M 39/105

(57) ABSTRACT

An apparatus for forming a catheter includes a catheter shaft and a hub including at least one connector for irremovably connecting the catheter shaft to the hub. The catheter shaft may engage one or more push connectors captured within a passage in the hub. Related are methods of manufacturing a catheter hub, including connecting first and second generally symmetrical halves of a hub over a connector, and then inserting a catheter shaft into the connector, such as by pushing it through a ring with inwardly directed fingers to form a secure locking engagement.

### BACKGROUND OF THE INVENTION

One current method of manufacturing a catheter, such as one including an inflatable medical balloon at a distal end, involves using an adhesive to bond a proximal end of catheter shaft to a hub. While this approach is in widespread practice and long-accepted, limitations exist.

For instance, leakage or instability may result if proper precautions are not taken during the process of applying the adhesive. Manufacturing time is also relatively high due to the requirement for the adhesive to cure adequately. Thus, the overall expense associated with manufacturing such a catheter can be high.

A need is thus identified for a catheter hub that allows for a quick and secure connection to the catheter shaft in a reliable manner, preferably without the use of adhesives. The hub would be easy to assemble and would avoid issues with leakage that might result if an adhesive is not properly applied. Overall, a significant improvement in reliability and ease of manufacture would be realized, with a concomitant reduction in cost.

### SUMMARY OF THE INVENTION

An object of the disclosure is to provide a catheter hub that allows for a quick and secure connection to the catheter shaft in a reliable manner, preferably without the use of adhesives. The hub uses one or more connectors, such as push connectors, that receive the shaft in one direction, and prevent withdrawal in the opposition direction. The hub would thus be easy to assemble, and may further include seals to avoid issues with leakage that might result if an adhesive is not properly applied. Overall, a significant improvement in reliability and ease of manufacture is realized, along with a concomitant reduction in cost.

According to a first aspect of the disclosure, an apparatus for forming a catheter is provided. The apparatus includes a catheter shaft and a hub including at least one connector for irremovably connecting the catheter shaft to the hub (meaning it cannot be removed without destruction or damage to the associated parts; for example, it cannot be removed simply by pulling the shaft in a distal direction with a similar force used to insert the shaft into the hub in a proximal direction during assembly).

In one embodiment, the at least one connector comprises a push connector captured within a passage of the hub. The push connector may comprise a ring including a plurality of inwardly directed fingers for engaging the shaft. A retainer may be provided for engaging the ring to prevent movement relative to the hub.

The at least one connector may comprise a first connector for connecting to a first proximal portion of the catheter shaft within the hub and further including a second connector for connecting to a second portion of the catheter shaft distally of the first proximal portion and still within the hub. The proximal portion of the catheter shaft may include a single lumen and the second portion of the shaft includes a dual lumen. The hub may comprise a first port in communication with a first lumen of the proximal portion of the catheter shaft and a second port in communication with a second lumen of the second portion of the catheter shaft. The first connector may have a larger diameter than the second connector, and the hub may comprise two generally symmetrical halves.

According to a further aspect of the disclosure, an apparatus for connecting to a catheter shaft for forming a catheter. The apparatus includes a hub including an internal passage including at least one push connector for connecting the shaft to the hub.

In one embodiment, the at least one connector comprises a push connector captured within a passage of the hub. The push connector further comprises a ring including a plurality of inwardly directed fingers for engaging the shaft. A retainer is also provided for retaining the ring to prevent movement relative to the hub.

The at least one connector may comprise a first connector for connecting to a proximal portion of the shaft within the hub. A second connector may also be provided, such as for connecting to a different (e.g., distal) portion of the shaft within the hub. The hub may comprise a first port for communicating with a first lumen of the proximal portion of the shaft and a second port for communicating with a second lumen of the second portion of the shaft. The first connector may have a larger diameter than the second connector, and the hub may comprise two generally symmetrical halves.

Still a further aspect of the disclosure pertains to an apparatus for forming a catheter. The apparatus comprises a catheter shaft, and a hub mechanically connected to the catheter shaft without using an adhesive. In one example, the hub comprises a push connector for connecting to the shaft.

Yet another aspect of the disclosure relates to a method for manufacturing a catheter assembly, which method includes inserting a catheter shaft into a hub including a connector for irremovably connecting the shaft to the hub. The method may further include the step of connecting first and second generally symmetrical halves of the hub over the connector. In one example, the inserting step comprises inserting the catheter shaft in a proximal direction through the connector to engage a retaining element for engaging the catheter shaft and preventing removal from the hub in a distal direction.

Another aspect of this disclosure is a method for manufacturing a catheter assembly including the steps of connecting first and second halves of a hub over a connector, and inserting a catheter shaft into the connector. The inserting step may comprise inserting the catheter shaft in a proximal direction through the connector to engage a retaining element for retaining the catheter shaft and preventing removal from the hub in a distal direction. The method may further comprise the step of providing first and second generally symmetrical halves of the hub.

### BRIEF DESCRIPTION OF THE DRAWINGS

A better understanding of the above and further advantages of the present disclosure results from referring to the following description in conjunction with the accompanying drawings in which:

FIG. 1 is a perspective view of a catheter hub;

FIG. 2 is a perspective view of the catheter hub of FIG. 1, irremovably connected to a catheter shaft by one or more connectors;

FIG. 3 is an enlarged cross-sectional, partially cutaway view of a distal end of the hub including one connector for the shaft;

FIG. 4 is a side view of the catheter hub of FIG. 1;

FIG. 5 is an enlarged, partially cutaway side view of the catheter hub of FIG. 4;

FIG. 6 is an enlarged, partially cutaway, cross-sectional side view of the catheter hub of FIG. 4; and

FIG. 7 is an exploded view of the catheter hub, illustrating the manner in which two generally symmetrical halves may be interconnected to capture the push connectors for receiving the catheter shaft in an irremovable manner.

The dimensions of some of the elements may be exaggerated relative to other elements for clarity or several physical components may be included in one functional block or element. Further, sometimes reference numerals may be repeated among the drawings to indicate corresponding or analogous elements. Moreover, some of the parts depicted in the drawings may be combined into a single function.

### DETAILED DESCRIPTION

In the following detailed description, numerous specific details are set forth to provide a thorough understanding of the presently disclosed invention(s). The disclosed embodiments may be practiced without these specific details. In other instances, well-known methods, procedures, components, or structures may not have been described in detail so as not to obscure the disclosed inventive concepts.

The invention of this disclosure is not limited in its application to the details of construction and the arrangement of the components set forth in the following description or illustrated in the drawings. The inventive concepts disclosed are capable of other embodiments or of being practiced or carried out in various ways. It is also to be understood that the phraseology and terminology employed herein are for the purpose of description and should not be regarded as limiting.

Certain features of the disclosed embodiments that are, for clarity, described in the context of separate embodiments, may also be provided in combination in a single embodiment. Conversely, various features of the invention that are, for brevity, described in the context of a single embodiment, may also be provided separately or in any suitable subcombination.

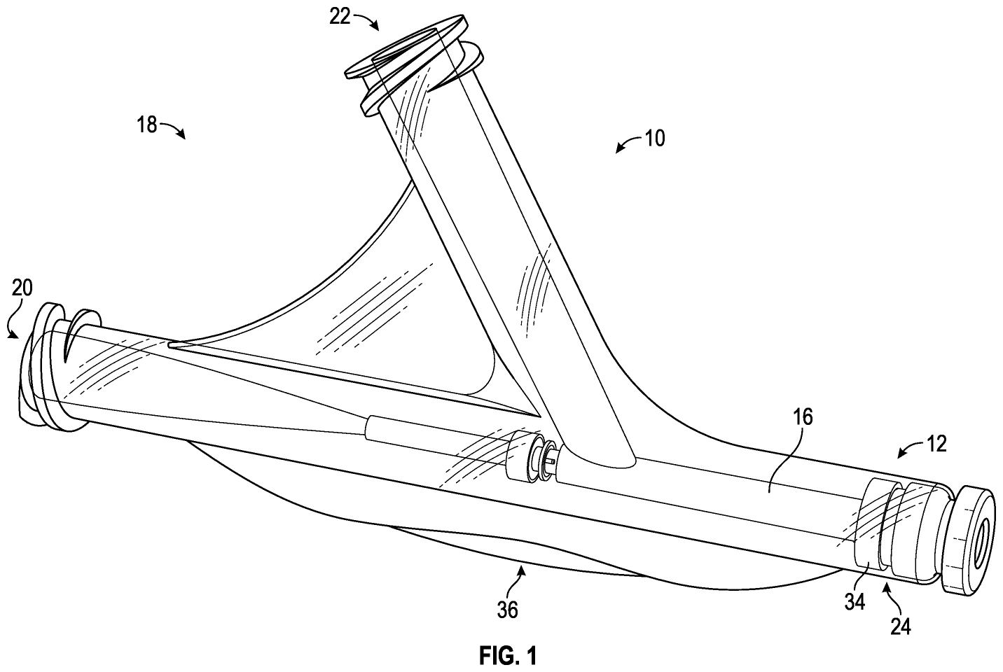

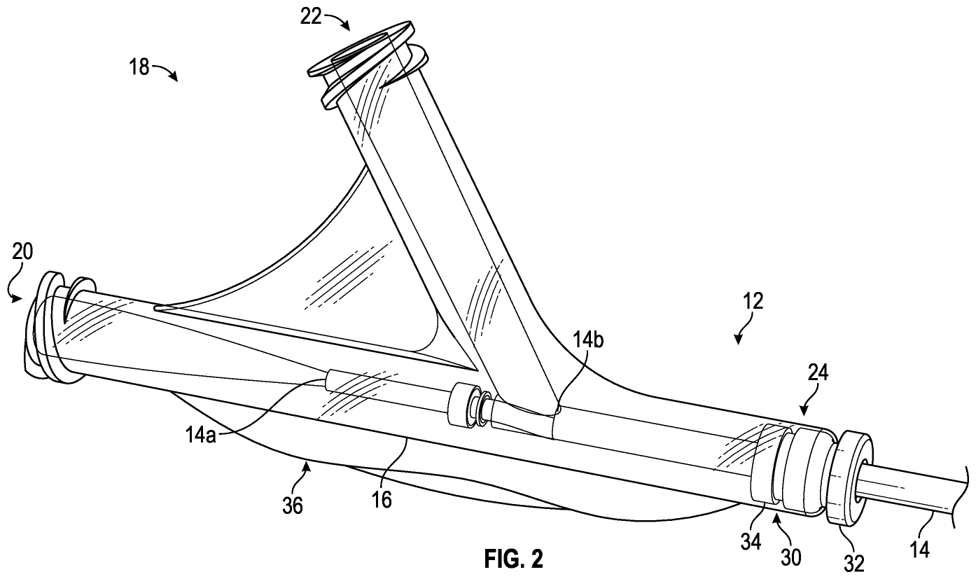

With reference to FIGS. 1 and 2, a catheter hub 10 is illustrated. In this example, the hub 10 includes a distal end 12 with a port adapted for receiving a catheter shaft 14 within an interior passage 16. The interior passage 16 is in communication with one or more ports adjacent a proximal end 18, such as for example a first port 20 for receiving a guidewire (not shown) for passing through a first lumen 14a in the catheter shaft 14. A second port 22 of the hub 10 (which may extend at an angle and form a “Y” shape, as illustrated) is provided for supplying a fluid to a second lumen 14b in this shaft 14, such as for inflating a medical balloon (not shown) attached to a distal end of the shaft 14. The ports 20, 22 may also include suitable structures for forming a connection with an external structure, such as a helical “luer” thread, as shown. While two ports 20, 22 is conventional, the hub 10 may include only a single port, or more than two ports (such as three, four, five, or more ports), depending on the particular application (e.g., in a rapid exchange configuration, where the guidewire enters through a distal port in the shaft 14, only a single port may be present in the hub).

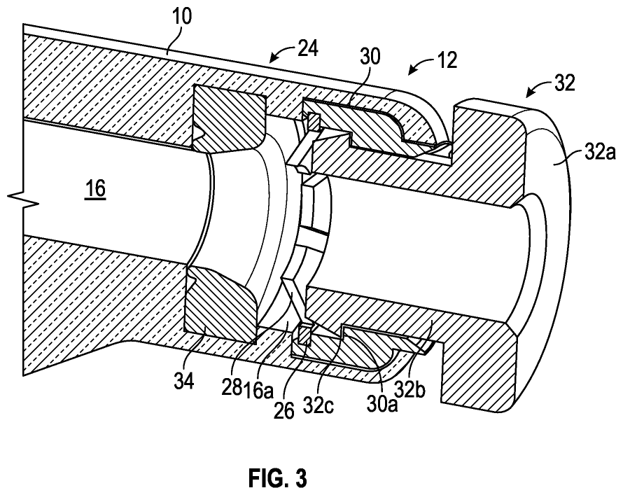

According to a first aspect of the disclosure, and with further reference to FIG. 3, the hub 10 may include at least one connector 24 for irremovably connecting the catheter shaft 14 to the hub 10. In the illustrated embodiment, the connector 24 is a mechanical arrangement in the form of a so-called “push” connector, which is captured within the passage 16 of the hub 10, such as at the distal end 12 as shown.

In the illustrated example, the connector 24 comprises a retaining element in the form of a ring 26 including a plurality of inwardly directed, radially extending fingers 28. The inner ends of the fingers 28 are arranged for engaging the outer surface of the shaft 14 when passed therethrough. In particular, the fingers 28 each have a radially inwardly directed end and extend radially a sufficient distance to engage an outer surface of the shaft 14, which has an outer diameter exceeding the inner extent of the fingers so as to create an engagement.

The ring 26 may be held in the passage 16, or in particular in an oversized portion forming a chamber 16a in the proximal end of the hub 10, by a retainer 30. The retainer 30 may be connected to the hub 10 and, in particular, to a collar 32. The collar 32 provides a smooth transition for the shaft from inside the hub 10 to the outside environment.

In the illustrated example, the collar 32 includes an oversized portion 32a external to the hub 10, and an elongated, tubular portion 32b extending into the hub into engagement with the fingers 28 to cause the same to bias in a proximal direction for purposes of engaging an outer surface of the shaft 14. The tubular portion 32b may include an annular ledge 32c for engaging a corresponding annular ledge 30a of the retainer 30. At least the tubular portion 32b may be formed of a resilient material to allow for the collar 32 to be pushed through the retainer 30 in a proximal direction, but then captured against movement in a distal direction. In this position, the proximal end of the tubular portion 32b may guard against any backbending of the fingers 28 and thus aid in resisting any pulling force applied to the shaft 12.

An annular seal 34 may also be provided, such as around a portion of the shaft 12 in the illustrated example. This seal 34 is provided for sealing the passage 16 to prevent fluid ingress or egress. As shown, the seal 34 may be captured in the chamber 16a at the distal end of the passage 16 by the presence of the retainer 30.

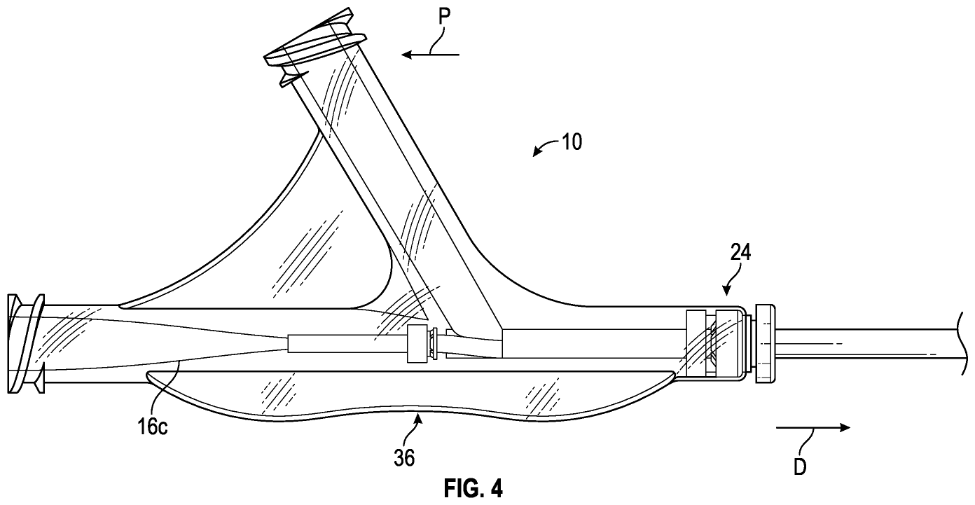

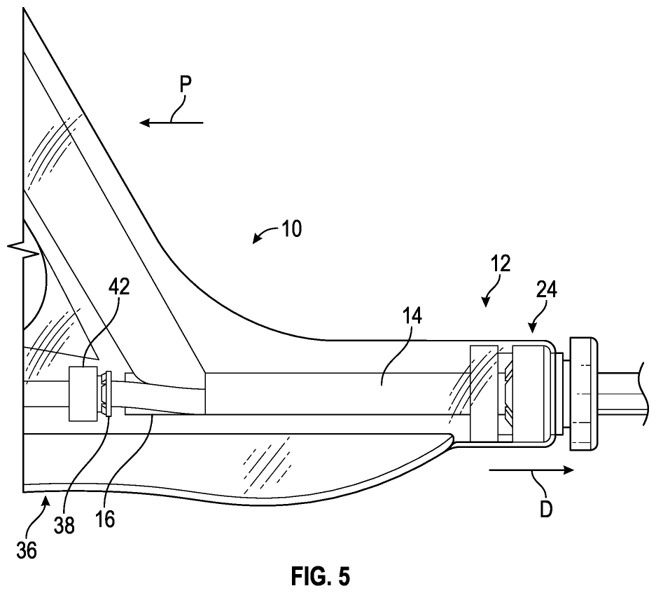

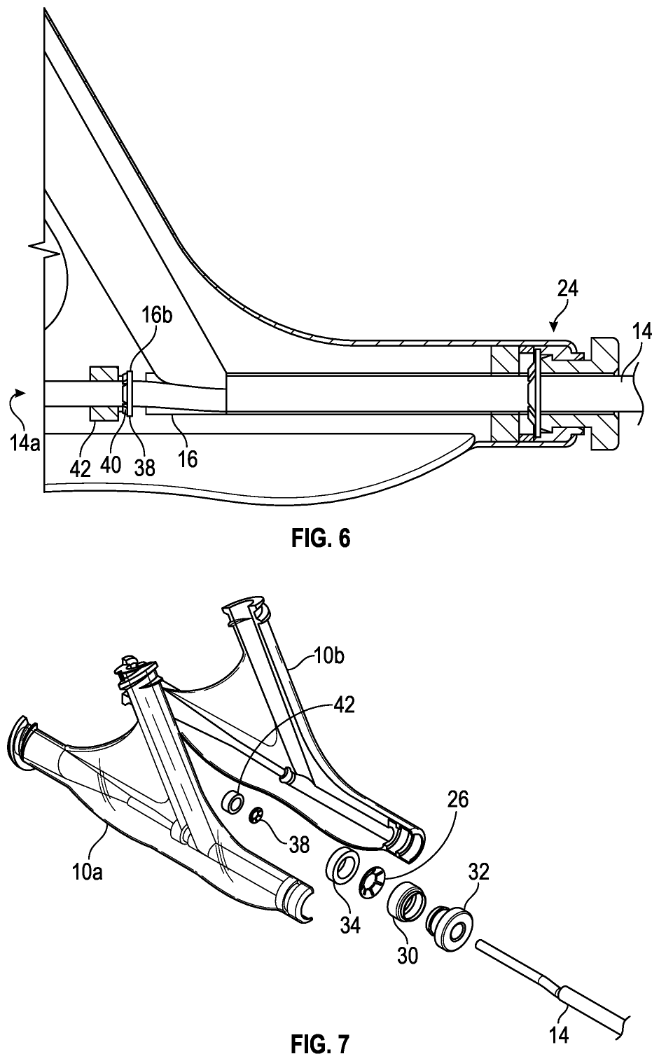

As mentioned above, the shaft 14 may include a “dual” lumen arrangement, with both guidewire and inflation lumens 14a, 14b. In such case, the inflation lumen 14a may terminate at or near a junction between the passage 16 and the second port 22 for delivering the inflation fluid from a source, such as an indeflator (not shown) connected to the second port 22. The shaft 14 may be cut or skived, and the other lumen 14a only may extend proximally within the passage 16 to communicate with the first port 20 for receiving a guidewire. As shown in FIGS. 4 and 5, a second connector 36 may be provided in the passage for forming a second connection for further irremovably connecting the catheter shaft 14 to the hub 10.

In the illustrated embodiment, this second connector 36 is a mechanical arrangement in the form of a so-called “push” connector, which is captured within the passage 16 of the hub 10, such as along an intermediate portion of the passage. In the illustrated example, the second connector 36 comprises a ring 38 including a plurality of inwardly directed, radially extending fingers 40 for engaging the outer surface of the shaft 14 when passed therethrough. As can be appreciated, because the diameter of the shaft 14 is smaller along the portion including the single lumen 14a, the second connector 36 may be adapted to engage this portion of the shaft, such as by being made proportionally smaller than the first connector 24.

The second connector 36 may be captured and held within the hub 10, such as within an oversized portion, or chamber 16b, of the passage 16. In this way, the fingers 40 once in engagement with the shaft 14 as a result of it being pushed therethrough, are biased proximally, and cannot move distally. In this manner, a secure mechanical connection is formed. An annular seal 42 may also be provided proximally of the connector 36 for sealing against the ingress or egress of fluid, including as a result of being introduced into the downstream port 22.

The hub 10 may be manufactured to include the one or more connectors 24, 36 in a variety of ways. In one example, the body of the hub 10 may be formed (e.g., molded) of a polymer material in generally symmetrical (Y-shaped) halves (see halves 10a, 10b in FIG. 7). The connectors 24 (ring 26), 36 (ring 38) may be pre-positioned in the corresponding portions of chambers 16a, 16b of the passage 16 (along with the retainer 30 and seals 34, 42) associated with one of the halves. The halves are then mated together and sealed to each other, such as by welding, such as along a seam, mechanical fastening, or the like to form the unitary hub 10 including the connectors 24, 36 for receiving the shaft 14 when inserted into a distal end of the hub 10 in a proximal direction. Interference or snap-fit arrangements could also be used to connect the mating halves of the hub 10. Alternatively, one or more of the connectors 24, 36, the seals 34, 42, and retainer 30 (if applicable) may be located in a mold cavity, along with suitable inserts for forming the passage 16 and ports 20, 22, and a material introduced into the mold to form the hub 10.

As can be appreciated, using this arrangement allows for the proximal end of the shaft 14 to be introduced into the hub 10 and mechanically captured in place. Specifically, in the arrangement shown in FIGS. 4 and 5, the shaft 14 is passed in a proximal direction P through the collar 32, the connector 24, and the seal 34, as well as connector 36 and seal 42, until the portion with the single lumen 14a “bottoms out” against a stop in the internal passage 16 (which may include a conical proximal portion 16c, as shown in FIG. 4). This portion of the shaft 14 is thereby captured in place because of engagement with the radially inward ends of the fingers 40 of the second, smaller connector 36, which has a smaller diameter than the other, first connector 24.

The second portion of the shaft 14 distal of the single lumen portion and still within the hub 10 including both lumens 14a, 14b is likewise captured in place by engagement with the radially inward ends of the fingers 28 of the first, larger connector 36 at the proximal end 12 of the hub 10. In this position, the opening of the inflation lumen 14b is located so as to be in communication with port 22 for supplying inflation fluid to an associated balloon on a distal end of the shaft 14. Because of the engagement with fingers 28, 40, pulling of the shaft 14 in the distal direction D is not possible, and the shaft 12 thus remains securely mechanically attached to the hub 10 in a fully sealed arrangement, even without the use of adhesives.

This disclosure also pertains to a method of manufacturing a catheter assembly including a hub. The method may involve connecting first and second generally symmetrical halves of the hub 10 (e.g., halves 10a, 10b in FIG. 7) which may be brought together over one or more connectors (including rings 26, 38, seals 34, 42, and retainer 30 for receiving collar 32) arranged as per the above disclosure. The halves 10a, 10b once brought together may be connected, such as by using an adhesive or mechanical fastener. The method may further include inserting a catheter shaft 14 into the hub 10 once formed, which may comprise inserting the catheter shaft 14 in a proximal direction to engage a retaining element, such as ring 26 or 38, for retaining the catheter shaft 14 and preventing removal from the hub 10 in a distal direction. This establishes a secure mechanical connection, potentially without the use of any adhesive.

Summarizing, this disclosure may be considered to relate to the following items:
1. An apparatus for forming a catheter, comprising:
   a catheter shaft; and
   a hub including at least one connector for irremovably connecting the catheter shaft to the hub.
2. The apparatus according to item 1, wherein the at least one connector comprises a push connector captured within a passage of the hub.
3. The apparatus according to item 2, wherein the push connector comprises a ring including a plurality of inwardly directed fingers for engaging the shaft.
4. The apparatus according to item 3, further including a retainer for engaging the ring to prevent movement relative to the hub.
5. The apparatus according to any of items 1-4, wherein the at least one connector comprises a first connector for connecting to a proximal portion of the catheter shaft within the hub and further including a second connector for connecting to another portion of the catheter shaft distal of the proximal portion and within the hub.
6. The apparatus according to item 5, wherein the proximal portion of the catheter shaft includes a single lumen and the other portion of the shaft includes a dual lumen.
7. The apparatus according to item 5, wherein the hub comprises a first port in communication with a first lumen of the proximal portion of the catheter shaft and a second port in communication with a second lumen of another portion of the catheter shaft.
8. The apparatus according to item 5, where the first connector has a larger diameter than the second connector.
9. The apparatus according to any of items 1-8, wherein the hub comprises two generally symmetrical halves.
10. An apparatus for connecting to a catheter shaft for forming a catheter, comprising:
   a hub including an internal passage including at least one push connector for connecting the shaft to the hub.
11. The apparatus according to item 10, wherein the at least one connector comprises a push connector captured within a passage of the hub.
12. The apparatus according to item 11, wherein the push connector comprises a ring including a plurality of inwardly directed fingers for engaging the shaft.
13. The apparatus according to item 12, further including a retainer for retaining the ring to prevent movement relative to the hub.
14. The apparatus according to any of items 10-13, wherein the at least one connector comprises a first connector for connecting to a proximal portion of the shaft within the hub and further including a second connector for connecting to another portion of the shaft within the hub distal of the proximal portion.
15. The apparatus according to item 14, wherein the hub comprises a first port for communicating with a first lumen of the proximal portion of the shaft and a second port for communicating with a second lumen of another portion of the shaft distal of the proximal portion and within the hub.
16. The apparatus according to item 14, where the first connector has a larger diameter than the second connector.
17. The apparatus according to item 14, wherein the hub comprises two generally symmetrical halves.
18. An apparatus for forming a catheter, comprising:
   a catheter shaft; and
   a hub mechanically connected to the catheter shaft without using an adhesive.
19. The apparatus of item 18, wherein the hub comprises a push connector for connecting to the shaft.
20. The apparatus of any of claims 1-19, further including one or more annular seals within the hub, preferably surrounding the catheter shaft.
21. A method for manufacturing a catheter, comprising:
   inserting a catheter shaft into a hub including a connector for irremovably connecting the shaft to the hub.
22. The method of item 21, further including the step of connecting first and second generally symmetrical halves of the hub over the connector.
23. The method of item 21 or item 22, wherein the inserting step comprises inserting the catheter shaft in a proximal direction through the connector to engage a retainer for engaging the catheter shaft and preventing removal from the hub in a distal direction.
24. A method for manufacturing a catheter, comprising:
   connecting first and second halves of a hub over a connector; and
   inserting a catheter shaft into the connector.
25. The method of item 24, wherein the inserting step comprises inserting the catheter shaft in a proximal direction through the connector to engage a retainer for retaining the catheter shaft and preventing removal from the hub in a distal direction.
26. The method of item 23 or item 24, further comprising the step of providing first and second generally symmetrical halves of the hub.

As used herein, the following terms have the following meanings: “A”, “an”, and “the” as used herein refers to both singular and plural referents unless the context clearly dictates otherwise. By way of example, “a compartment” refers to one or more than one compartment.

“About,” “substantially,” “approximately,” or “generally” as used herein referring to a measurable value, such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed. The term “generally” as used in connection with symmetrical is intended to connote a high degree of symmetry, such as in relation to perimeter, but not necessarily exact symmetry (such that one part may have, for example, a different profile, added projection, embossments or the like and still be considered generally symmetrical).

“Comprise”, “comprising”, and “comprises” and “comprised of” as used herein are synonymous with “include”, “including”, “includes” or “contain”, “containing”, “contains” and are inclusive or open-ended terms that specifies the presence of what follows, e.g., component or the like does not exclude or preclude the presence of additional, nonrecited components, features, element, members, steps, known in the art or disclosed therein.

Although the invention is described in conjunction with specific embodiments, many alternatives, modifications, and variations will be apparent to those skilled in the art. Accordingly, this disclosure embraces all such alternatives, modifications, and variations that fall within the spirit and scope of the appended claims. For example, a small, shielded skive blade could optionally be added to the distal port of the hub 10 to simultaneously skive and seal the shaft 14 onto the hub. The hub 10 is also show as being formed of a transparent material, but could have any coloring, applied logos or other indicia, or varying degrees of opacity. It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment may also be illustratively described and presented in the context or format of a plurality of separate embodiments.

1. An apparatus for forming a catheter assembly, comprising:
a catheter shaft; and
a hub including at least one connector for irremovably connecting the catheter shaft to the hub.

2. The apparatus according to claim 1, wherein the at least one connector comprises a push connector captured within a passage of the hub.

3. The apparatus according to claim 2, wherein the push connector comprises a ring including a plurality of inwardly directed fingers for engaging the shaft.

4. The apparatus according to claim 3, the hub further including a retainer for engaging the ring to prevent movement relative to the hub.

5. The apparatus according to claim 1, wherein the at least one connector comprises a first connector for connecting to a first proximal portion of the catheter shaft within the hub and further including a second connector for connecting to a second portion disposed distally of the first proximal portion of the catheter shaft within the hub.

6. The apparatus according to claim 5, wherein the proximal portion of the catheter shaft includes a single lumen and the second portion of the shaft includes a dual lumen.

7. The apparatus according to claim 5, wherein the hub comprises a first port in communication with a first lumen of the proximal portion of the catheter shaft and a second port in communication with a second lumen of the second portion.

8. The apparatus according to claim 5, where the first connector has a larger diameter than the second connector.

9. The apparatus according to claim 1, wherein the hub comprises two generally symmetrical halves.

10. An apparatus for connecting to a catheter shaft for forming a catheter, comprising:
a hub including an internal passage including at least one push connector for connecting the shaft to the hub.

11. The apparatus according to claim 10, wherein the at least one connector comprises a push connector captured within a passage of the hub.

12. The apparatus according to claim 11, wherein the push connector comprises a ring including a plurality of inwardly directed fingers for engaging the shaft.

13. The apparatus according to claim 12, the hub further including a retainer for retaining the ring to prevent movement relative to the hub.

14. The apparatus according to claim 10, wherein the at least one connector comprises a first connector for connecting to a first proximal portion of the shaft within the hub and further including a second connector for connecting to a second portion of the shaft disposed distally of the first proximal portion of the shaft within the hub.

15. The apparatus according to claim 14, wherein the hub comprises a first port for communicating with a first lumen of the proximal portion of the shaft and a second port for communicating with a second lumen of the second portion of the shaft.

16. The apparatus according to claim 14, where the first connector has a larger diameter than the second connector.

17. The apparatus according to claim 14, wherein the hub comprises two generally symmetrical halves.

18. An apparatus for forming a catheter, comprising:
a catheter shaft; and
a hub mechanically connected to the catheter shaft without using an adhesive.

19. The apparatus of claim 18, wherein the hub comprises a push connector for connecting to the shaft.

20. The apparatus of claim 18, further including one or more annular seals for sealing the catheter shaft within the hub.

21-25. (canceled)
